(19) United States
(12) Patent Application Publication
(10) Pub. No.: US 2026/0335139 A1
(43) Pub. Date: Oct. 29, 2026

(54) Banded Earmuffs with Lightweight Headband

(71) Applicant: Milwaukee Electric Tool Corporation, Brookfield, WI (US)

(72) Inventors: Brady James Schindler, Oconomowoc, WI (US); Frank C. Pizzitola, Jackson, WI (US)

(21) Appl. No.: 19/576,689

(22) Filed: Mar. 24, 2026

Related U.S. Application Data
(60) Provisional application No. 63/793,889, filed on Apr. 24, 2025.

(51) Int. Cl. A61F 11/14

(52) U.S. Cl. CPC A61F 11/14

(57) ABSTRACT

A banded earmuff with a headband, a first earmuff, and a second earmuff is provided. The headband includes at least one opening defined along a length of the headband between a first end, a second end, a first outer arched segment, a second outer arched segment. The headband is configured to provide a compressive force to the first earmuff and the second earmuff to bias the first earmuff and the second earmuff towards each other.

### CROSS-REFERENCE TO RELATED PATENT APPLICATION

The present application claims the benefit of and priority to U.S. Provisional Application No. 63/793,889 filed on Apr. 24, 2025, which is incorporated herein by reference in its entirety.

### BACKGROUND OF THE INVENTION

The present invention relates generally to the field of personal protective equipment. The present invention relates specifically to banded earmuffs designs to cover a user's ears for hearing protection.

### SUMMARY OF THE INVENTION

One embodiment of the invention relates to a banded earmuff. The banded earmuff includes a headband, a first ear cup, and a second ear cup. The headband includes a first end, a second end opposite the first end, and a central portion extending between the first end and the second end. The central portion includes a first outer arched segment extending from the first end to the second end and a second outer arched segment opposite the first outer arched segment and extending from the first end to the second end. The second outer arched segment is spaced apart from the first outer arched segment. At least one opening is defined along a length of the headband between the first end, the second end, the first outer arched segment, and the second outer arched segment. The first ear cup is coupled to the first end. The second ear cup is coupled to the second end. The headband provides a compressive force to the first ear cup and the second ear cup to bias the first ear cup and the second ear cup towards each other.

Another embodiment of the invention relates to a hearing protector. The hearing protector includes a headband, a first earmuff, and a second earmuff. The headband includes a first end, a second end opposite the first end, a first outer arched segment extending along a length of the headband from the first end to the second end, and a second outer arched segment spaced apart from the first outer arched segment and extending along the length of the headband from the first end to the second end. An opening is defined between the first end, the second end, the first outer arched segment, and the second outer arched segment. The first earmuff is coupled to the first end of the headband. The first earmuff includes a first pad. The second earmuff is coupled to the second end of the headband. The second earmuff includes a second pad. The headband is configured to bias the first earmuff and the second earmuff towards the head of a user when the hearing protector is worn.

Another embodiment of the invention relates to a headband for protective hearing equipment. The headband includes a first end and a second end opposite the first end. A first outer arched segment extends from the first end to the second end. A second outer arched segment is opposite the first outer arched segment and extends from the first end to the second end. A first base segment is at the first end and extends between the first outer arched segment and the second outer arched segment. A second base segment is at the second end and extends between the first outer arched segment and the second outer arched segment. An opening is defined between the first base segment, the second base segment, the first outer arched segment, and the second outer arched segment. The opening includes a first end terminating at the first base segment and a second end opposite the first end and terminating at the second base segment. At least a portion of the opening is uninterrupted between the first end of the opening and the second end of the opening.

Additional features and advantages will be set forth in the detailed description which follows and will be readily apparent to those skilled in the art from the description or recognized by practicing the embodiments as described in the written description and/or shown in the accompanying drawings. It is to be understood that both the foregoing general description and the following detailed description are exemplary.

The accompanying drawings are included to provide further understanding and are incorporated in and constitute a part of this specification. The drawings illustrate one or more embodiments and, together with the description, serve to explain principles and operation of the various embodiments. In addition, alternative exemplary embodiments relate to other features and combinations of features as may be generally recited in the claims.

### BRIEF DESCRIPTION OF THE DRAWINGS

This application will become more fully understood from the following detailed description, taken in conjunction with the accompanying figures, wherein like reference numerals refer to like elements in which:

FIG. 1 is a perspective view of banded earmuffs, according to an exemplary embodiment;

FIG. 2 is a left side view of the banded earmuffs of FIG. 1, according to an exemplary embodiment;

FIG. 3 is a detailed side view of a cup of the banded earmuffs of FIG. 1, according to an exemplary embodiment.

FIG. 4 is a perspective view of a headband for use with banded earmuffs, according to an exemplary embodiment;

FIG. 5 is a left side view of the headband of FIG. 4, according to the exemplary embodiment;

FIG. 6 is a back side view of the headband of FIG. 4, according to the exemplary embodiment;

FIG. 7 is a perspective view of banded earmuffs, according to an exemplary embodiment;

FIG. 8 is a left side view of the banded earmuffs of FIG. 7, according to an exemplary embodiment;

FIG. 9 is a back side view of the banded earmuffs of FIG. 7, according to an exemplary embodiment;

FIG. 10 is a cross-sectional view across the line 10-10 in FIG. 9, according to an exemplary embodiment;

FIG. 11 is a detailed perspective view of the headband of the banded earmuffs of FIG. 7, according to an exemplary embodiment;

FIG. 12 is an exploded view of the headband of FIG. 11, according to an exemplary embodiment; and

FIG. 13 is a detailed top view of a central band of the headband of FIG. 11, according to an exemplary embodiment.

### DETAILED DESCRIPTION

Referring generally to the figures, various embodiments of protective hearing equipment, such as banded earmuffs, are shown. Generally, earmuffs are used to protect a user's ears from excessive or loud noises, such as noises on construction sites or in other loud work environments. Conventional earmuffs include a solid headband coupled to a pair of cups (e.g., a left cup and a right cup). In the embodiments discussed herein, Applicant has found that providing a headband with one or more cut-outs formed along the length of the headband provides various advantages over conventional earmuffs. Specifically, Applicant has found that the cut-out designs discussed herein reduce the weight of the banded earmuffs, which may be more comfortable for users than conventional earmuffs, and provide sufficient clamping forced needed to form a secure fit around a user's head and protect a user's ears.

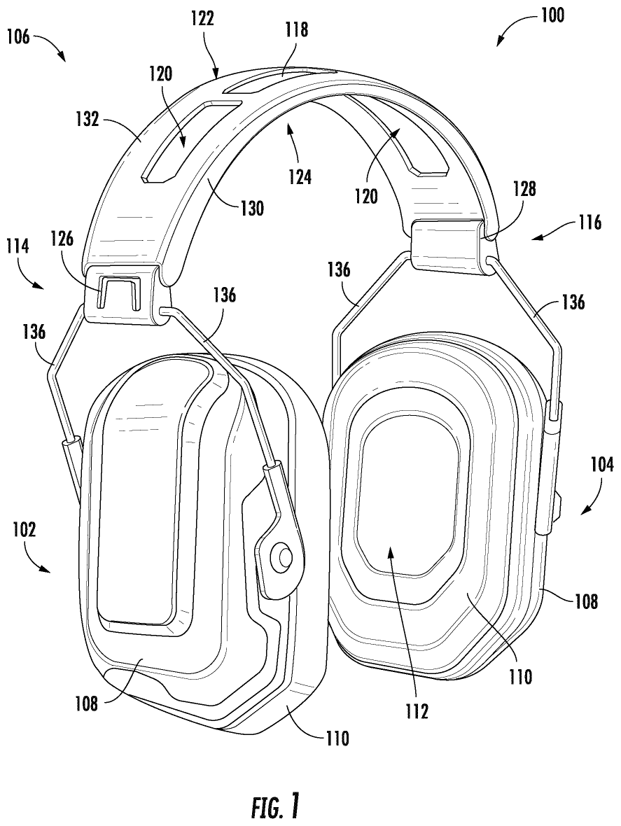

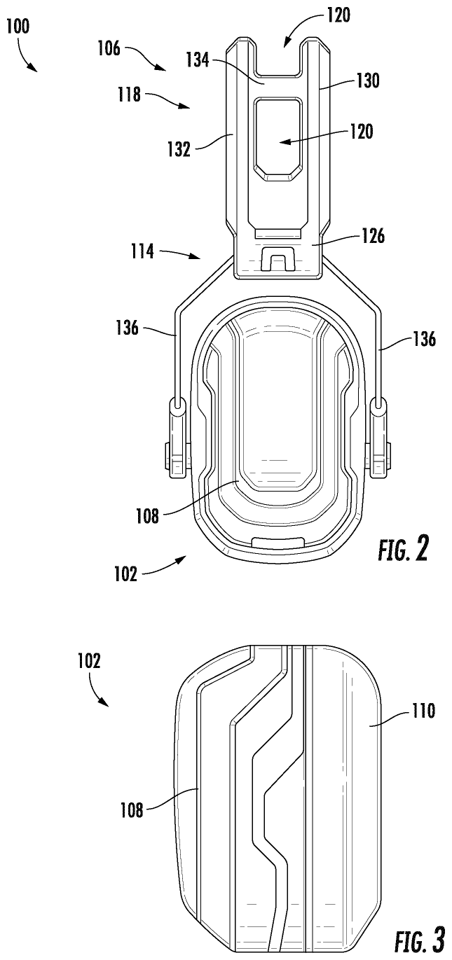

Referring to FIGS. 1-3, a hearing protector, shown as banded earmuffs 100 are provided. Banded earmuffs 100 include a first earmuff or first ear cup 102, a second earmuff or second ear cup 104, and a headband 106 extending between first ear cup 102 and second ear cup 104.

Ear cups 102 and 104 are configured to cover a user's ear when worn. Each ear cup 102 and 104 includes an outer shell 108 and a cushion or pad 110. Pads 110 are coupled to outer shells 108 and each define a recessed surface 112 configured to receive the ear of a user. Pads 110 are formed from sound-dampening materials, such as acoustic foam. Compressive forces provided by headband 106 bias cups 102 and 104 towards a user's head to create a secure fit around a user's ears when banded earmuffs 100 are worn.

Headband 106 includes a first end 114, a second end 116 opposite the first end 114, and a body or central portion 118 extending between first end 114 and second end 116. As shown, central portion 118 includes at least one opening or cut-out 120. Cut-outs 120 extends through headband 106 from a first or top side 122 to a second or bottom side 124. Cut-outs 120 are formed along a length of headband 106 measured between first end 114 and second end 116.

In particular, headband 106 includes a first base segment 126 located at first end 114 and a second base segment 128 located at second end 116. A first outer arched segment 130 and a second outer arched segment 132 extend from first end 114 to second end 116. Specifically, first outer arched segment 130 and a second outer arched segment 132 extend along the length of headband 106 between first base segment 126 and second base segment 128. First outer arched segment 130 and a second outer arched segment 132 extend substantially parallel to each other and are uninterrupted along the length of headband 106. Second outer arched segment 132 is spaced from first outer arched segment 130 and cut-outs 120 are positioned between first outer arched segment 130 and second outer arched segment 132. Cut-outs 120 are defined at least in part by first base segment 126, second base segment 128, first outer arched segment 130, and second outer arched segment 132.

First outer arched segment 130 and a second outer arched segment 132 are relatively narrow such that each arched segment has a width that is less than the width of headband 106. In a specific embodiment, first outer arched segment 130 has a width less than or equal to 33% of the width of headband 106, and second outer arched segment 132 has a width less than or equal to 33% of the width of headband 106.

Lateral segments 134 extend between first outer arched segment 130 and a second outer arched segment 132 to define cut-outs 120. Lateral segments 134 extend across the width of headband 106 in a direction substantially perpendicular to first outer arched segment 130 and a second outer arched segment 132. Lateral segments 134 separate cut-outs 120 from each other and define cut-outs 120 along with first base segment 126, second base segment 128, first outer arched segment 130, and second outer arched segment 132.

Headband 106 is coupled to ear cups 102 and 104 by support arms 136. As shown, two support arms 136 are coupled to first end 114 of headband 106. Support arms 136 extend between first base segment 126 and opposing sides of ear cup 102. Similarly, two support arms 136 are coupled to second end 116 of headband 106. Support arms 136 extend between second base segment 128 and opposing sides of ear cup 104.

Headband 106 provides a compressive force to first ear cup 102 and second ear cup 104 to bias first ear cup 102 and second ear cup 104 towards each other. Headband 106 provides a sufficient clamping force (e.g., a compressive force, biasing force) to bias cups 102, 104 towards a user's head to secure ear cups 102, 104 against a user's head and create a protective seal, while cut-outs 120 reducing the overall weight of the earmuffs 100. Applicant has found that if the weight is reduced, but a sufficient clamping force is not maintained then earmuffs 100 may shift during use and the seal between ear cups 102, 104 and a user's head may become less secure. In a specific embodiment, sufficient clamping force is at least 9.81 Newtons (N) of force. That is, the clamping force is equal to or greater than 9.81N.

When adjusting the weight of headband 106, the number or size of cut-outs 120, and/or the length of headband 106, a sufficient clamping force is maintained by adjusting the sectional moment of inertia measured along a cross-section of the headband 106. By maintaining a specific moment of inertia, the area of the cross-section can be adjusted by changing the thickness of headband 106, changing the width of cut-outs 120, and/or changing the length of headband 106. In various embodiments, the sectional moment of inertia is at least 0.63 mm⁴. In other various embodiments, the sectional moment of inertia is at most 0.7 mm⁴. In various embodiments, the length of headband 106 is in a range from 285 mm to 290 mm.

Further, headband 106 is designed to resist torsion, twisting, or bending. In particular, first outer arched segment 130 and second outer arched segment 132 are arched and bend in a direction towards the head of a user when banded earmuffs 100 are worn. In a specific embodiment, first outer arched segment 130 and second outer arched segment 132 provide sufficient spring force to secure banded earmuffs 100 on a user's head. In another specific embodiment, first outer arched segment 130 and second outer arched segment 132 resist torsion/bending in a direction along the width of headband 106. In another specific embodiment, lateral segments 134 are positioned between first outer arched segment 130 and second outer arched segment 132 along the length of headband 106 to further resist torsion, bending, or twisting in the width direction of headband 106.

In a specific embodiment, headband 106 is made from a metal material.

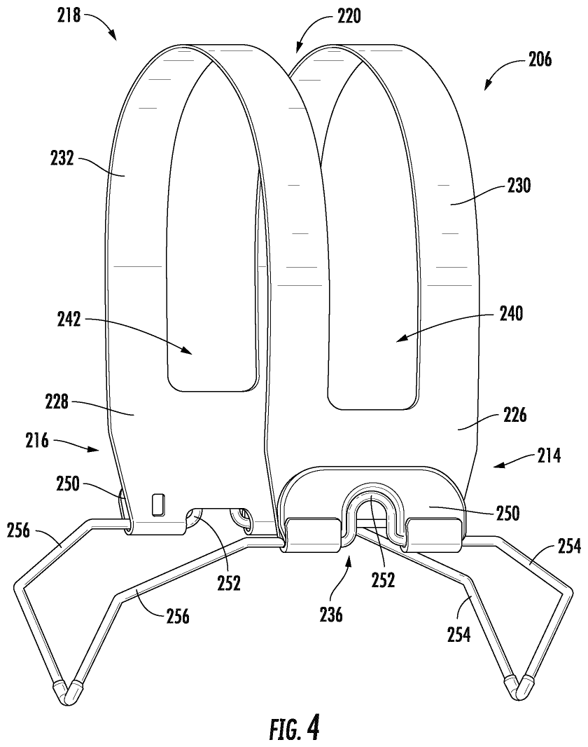

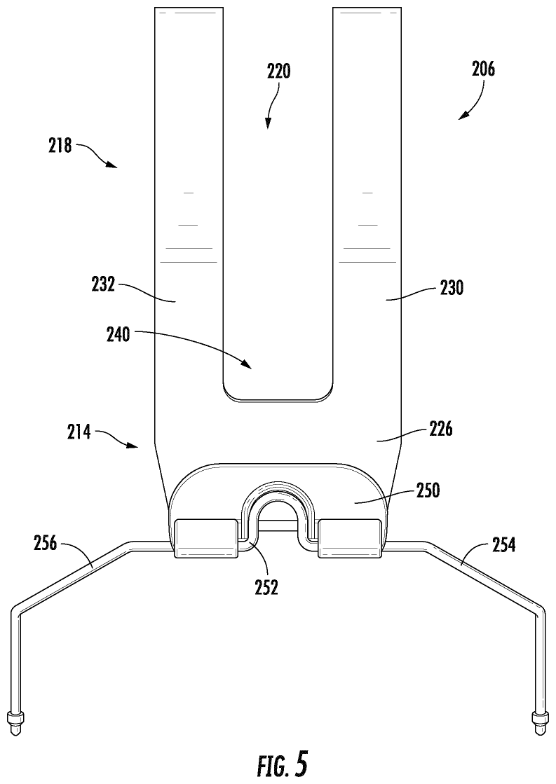

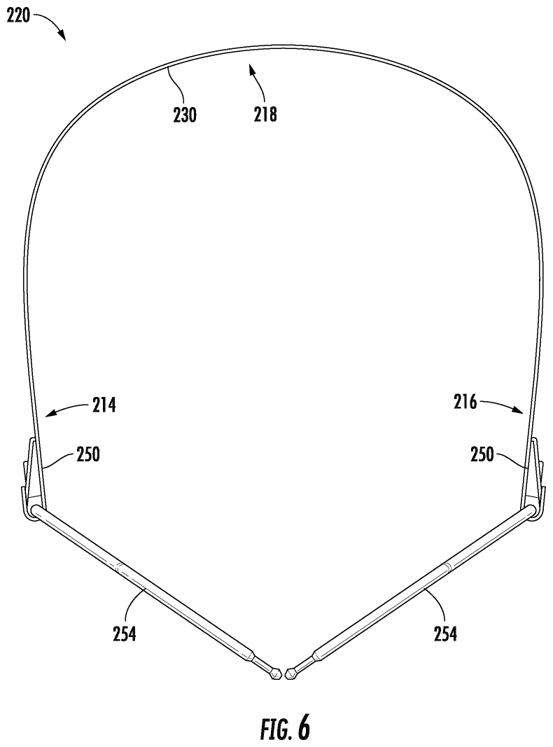

Referring to FIGS. 4-6, a headband 206 for use with hearing protectors or banded earmuffs, such as banded earmuffs 100, are shown. Headband 206 is substantially the same as headband 106, except for the differences discussed herein. In particular, headband 206 has one opening or cut-out 220. Headband 206 is made from a single, continuous, contiguous piece of material and cut-out 220 is made by stamping or punching-out a portion of headband 206.

Headband 206 includes a first end 214, a second end 216 opposite the first end 214, and a body or central portion 218 extending between first end 214 and second end 216. Cut-out 220 extends between first end 214 and second end 216. Cut-out 220 is defined between a first base segment 226, a second base segment 228, a first outer arched segment 230, and a second outer arched segment 232 of headband 206. Cut-out 220 is continuous and uninterrupted along the length of headband 206.

Cut-out 220 includes a first end 240 that terminates at first base segment 226 and a second end 242 opposite first end 240 that terminates a second base segment 228. At least a portion of cut-out 220 is uninterrupted between first end 240 and second end 142. As shown, cut-out 220 is continuous and uninterrupted along between first end 240 and second end 242. As shown, cut-out defines a rectangular shape and ends 240, 242 extend substantially straight between first outer arched segment 230 and second outer arched segment 232. In other embodiments, ends 240, 242 may be tapered or angled between first outer arched segment 230 and second outer arched segment 232.

As shown, a plurality of support arms 236 are coupled to first end 214 and second end 216 of headband 206 by catch or clip 250. In particular, headband 206 includes two support arms 236. Each support arm 236 is coupled to a clip 250 at opposing ends 214, 216 of headband. Support arms 236 each include a center portion 252 retained within clips 250 and a first arm 254 extending from center portion 252 in a first direction and a second arm 256 extending from center portion 252 in a second direction opposite the first direction.

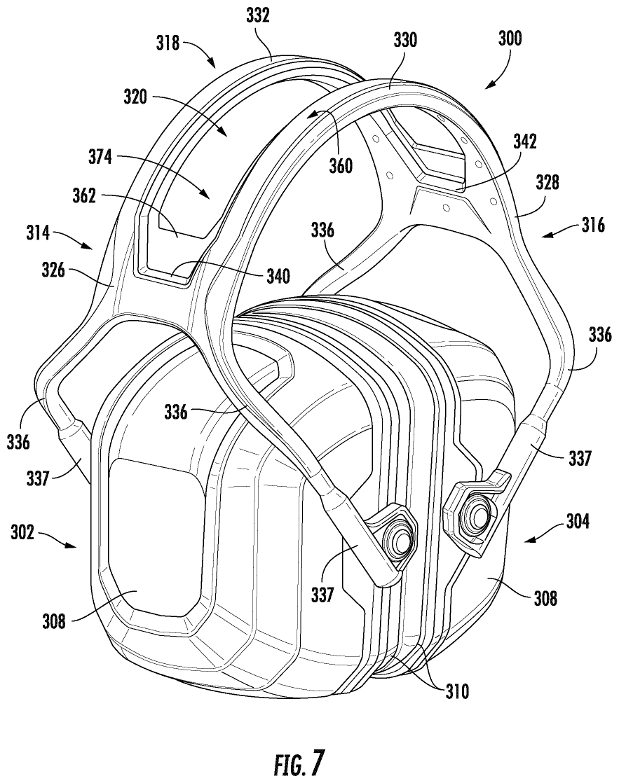

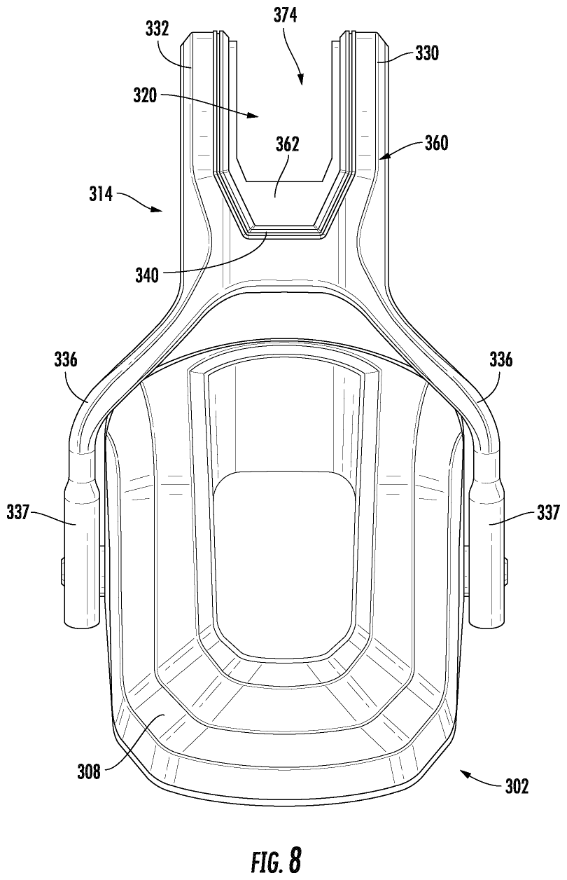

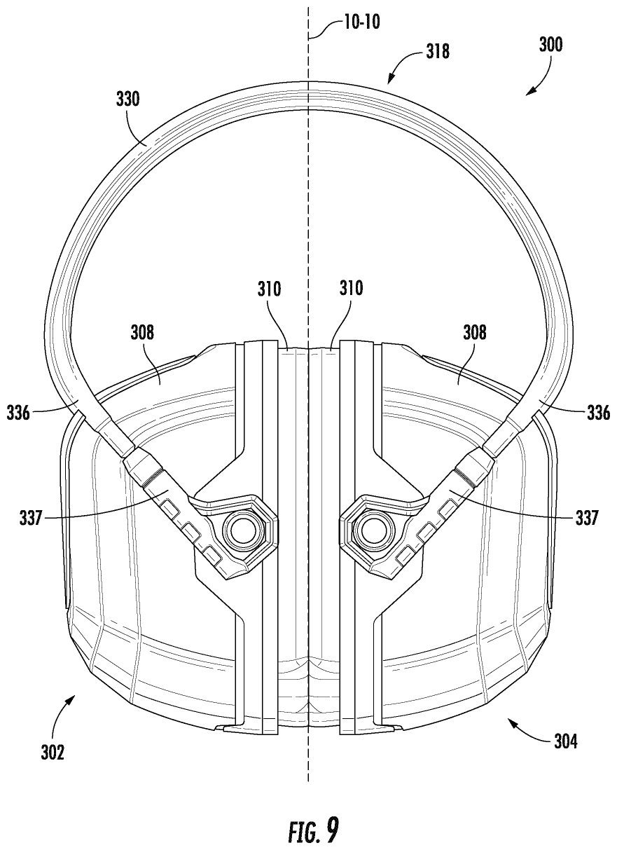

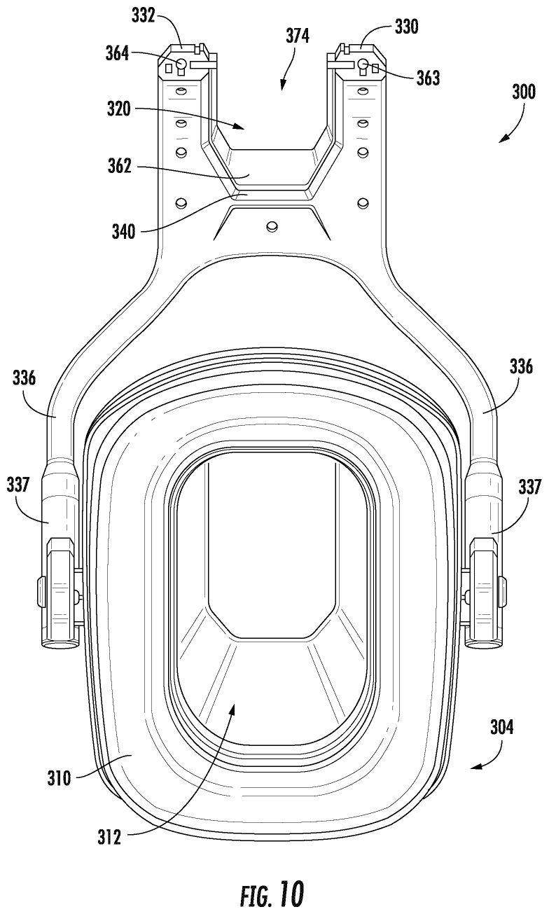

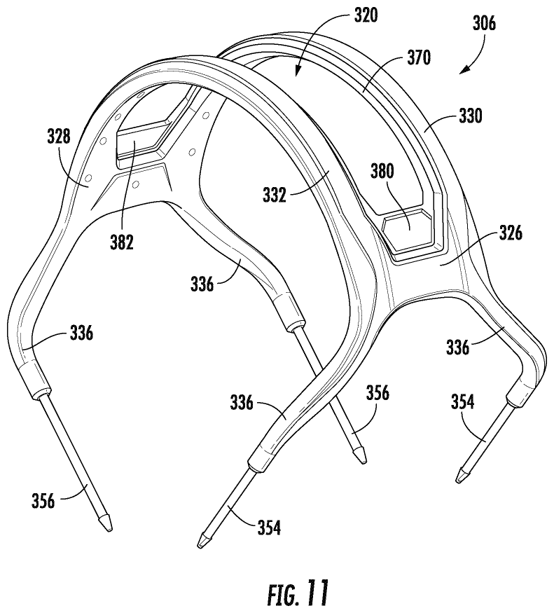

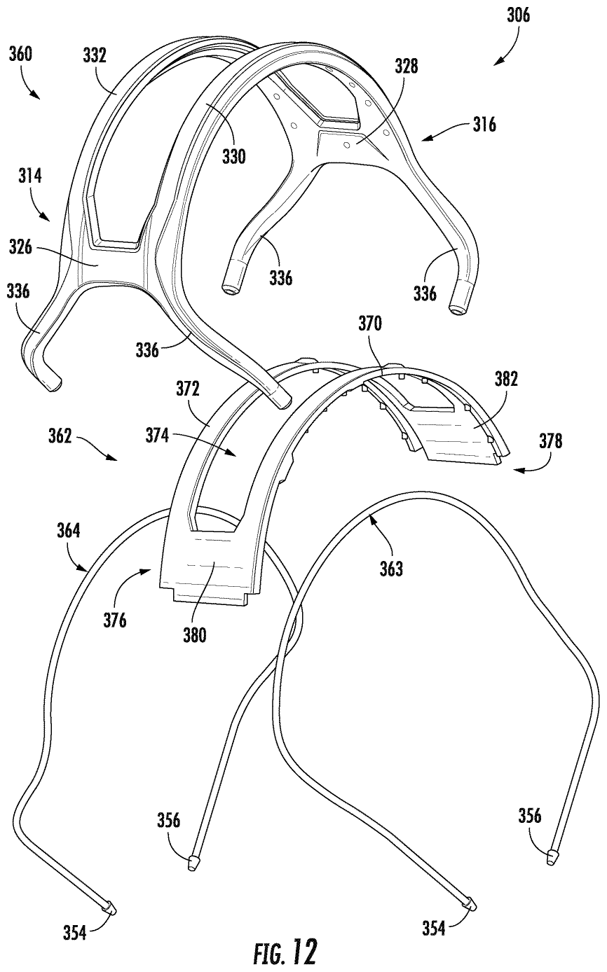

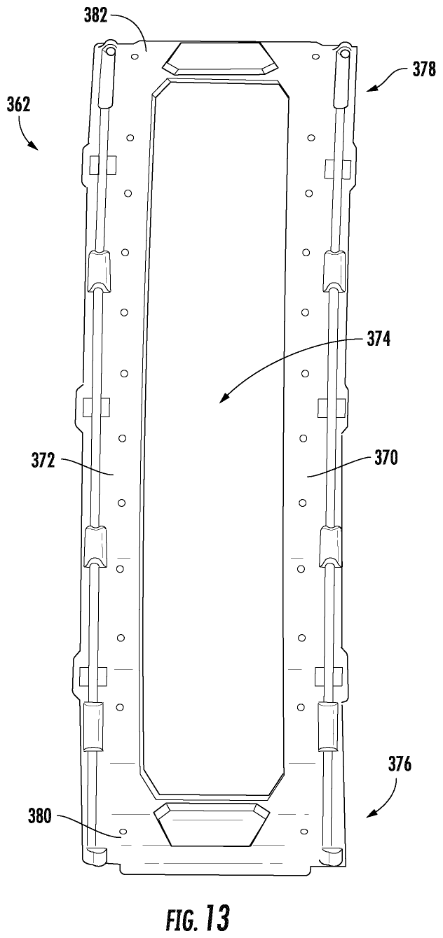

Referring to FIGS. 7-13, a hearing protector, shown as banded earmuffs 300 are provided. Banded earmuffs 300 are substantially the same as banded earmuffs 100, except for the differences discussed herein. Banded earmuffs 300 includes a first earmuff or first ear cup 302, a second earmuff or second ear cup 304, and a headband 306 extending between first ear cup 302 and second ear cup 304. First ear cup 302 and second ear cup 304, and headband 306 are substantially the same as first ear cup 102, except for the differences discussed herein. Headband 306 is substantially the same as headband 106 and headband 206, except for the differences discussed herein.

In particular, headband 306 includes a shell or outer frame 360, a center support or insert, shown as central band 362, and wire segments 363, 364. Frame 360, center band 362, and wire segments 363, 364 cooperate to provide a sufficient clamping force (e.g., a compressive force, biasing force) to bias cups 302, 304 towards and user's head. Specifically, wire segments 363, 364 primarily contribute to the biasing force. Central band 362 provides support to the wire segments 363, 364 and, which allows central band 362 to assist in providing the biasing force as well as reduce torsion, twisting, or bending of headband 306. Frame 360 surrounds a portion of central band 362 and wire segments 363, 364 to assist in coupling the wire segments 363, 364 to central band 362 and provide additional rigidity and support to the biasing force of headband 306.

As shown, frame 360 includes a first end 314, a second end 316 opposite the first end 314, and a body or central portion 318 extending between first end 314 and second end 316. A first opening 320 extends between first end 314 and second end 316. First opening 320 is defined between a first base segment 326, a second base segment 328, a first outer arched segment 330, and a second outer arched segment 332 of frame 360. Opening 320 extends uninterrupted along the length of frame from first base segment 326 to second base segment 328.

Opening 320 includes a first end 340 that terminates at first base segment 326 and a second end 342 opposite first end 340 that terminates a second base segment 328. First end 340 and second end 342 are tapered or angled such that the width of opening 320 at first end 340 and second end 342 is less than the width of opening 320 at a location between first end 340 and second end 342.

Central band 362 is coupled to frame 360 and extends between first base segment 326, second base segment 328, first outer arched segment 330, and second outer arched segment 332. Central band 362 is positioned such that it overlaps with at least a portion of opening 320. As shown, central band 362 overlaps with at least a first portion of first end 340 of opening 320 and a second portion of second end 342 of opening 320. A first wire segment 363 is coupled to a first side 370 of central band 362 and a second wire segment 364 is coupled to a second side 372 of central band 362 opposite first side 370. As shown, wire segments 363, 364 extend along and through at least a portion of the respective side 370, 372 of central band 362. In a specific embodiment, wire segments 363, 364 are retained along the respective sides 370, 372 of central band 362 via an interference fit or snap-fit. In another specific embodiment, channels are defined along the sides 370, 372 of central band 362 and wire segments 363, 364 are received and retained within the channels.

Wire segments 363, 364 are further retained within frame 360. First wire segment 363 is coupled to and retained within first outer arched segment 330 of frame 360, and second wire segment 364 is coupled to and retained within second outer arched segment 332 of frame 360. In a specific embodiment, frame 360 is made by overmolding such that frame is molded over wire segments 363, 364 and sides 370, 372 of central band 362.

Each wire segment 363, 364 includes a first end 354 and a second end 356. First ends 354 are configured to couple to first ear cup 302, and second ends 356 are configured to couple to second ear cup 304. Frame 360 extends along wire segments 363, 364 between first ends 354 and second ends 356. As shown, plurality of support arms 336 of frame 360 extend towards first ends 354 and second ends 356 along wire segments 363, 364. Ends of support arms 336 and first and second ends 354, 356 of wire segments 363, 364 are received and retained in side couplers 337. Side couplers 337 are mounted on and are coupled to first ear cup 302 and second ear cup 304.

Central band 362 includes a first end 376 and a second end 378 opposite first end 376. Central band 362 includes a first base portion 380 at first end 376 and a second base portion 382 at second end 378. First side 370 extends from first end 376 to second end 378, and more specifically from first base portion 380 to second base portion 382. Second side 372 extends from first end 376 to second end 378 opposite first side 370, and more specifically from first base portion 380 to second base portion 382. Second side 372 is space apart from first side 370. A second opening 374 is defined between first side 370, second side 372, first base portion 380, and second base portion 382 of central band 362. Second opening 374 is uninterrupted and continuous along the length of central band 362 from first base portion 380 to second base portion 382.

When coupled to frame 360, second opening 374 is positioned such that it overlaps with and is surrounded by first opening 320. In particular, second opening 374 is positioned between first base segment 326, second base segment 328, first outer arched segment 330, and second outer arched segment 332 of frame 360. As shown, the area of second opening 374 is smaller than the area of first opening 320. Second opening 374 is positioned between first end 340 and second end 342 of first opening 320. In this way, second opening 374 is nested within first opening 320. Applicant believes that the combination of frame 360 with a first opening 320 and central band 362 with a second opening 374 assists headband 306 in having a reduce weight while maintaining sufficient clamping force and reducing torsion, twisting, or bending of headband 306.

As shown, central band 362 has a thickness that is less than the thickness of frame 360.

It should be understood that the figures illustrate the exemplary embodiments in detail, and it should be understood that the present application is not limited to the details or methodology set forth in the description or illustrated in the figures. It should also be understood that the terminology is for the purpose of description only and should not be regarded as limiting.

Further modifications and alternative embodiments of various aspects of the disclosure will be apparent to those skilled in the art in view of this description. Accordingly, this description is to be construed as illustrative only. The construction and arrangements, shown in the various exemplary embodiments, are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Some elements shown as integrally formed may be constructed of multiple parts or elements, the position of elements may be reversed or otherwise varied, and the nature or number of discrete elements or positions may be altered or varied. The order or sequence of any process, logical algorithm, or method steps may be varied or re-sequenced according to alternative embodiments. Other substitutions, modifications, changes, and omissions may also be made in the design, operating conditions, and arrangement of the various exemplary embodiments without departing from the scope of the present disclosure.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred. In addition, as used herein, the article “a” is intended to include one or more component or element and is not intended to be construed as meaning only one.

For purposes of this disclosure, the term “coupled” means the joining of two components directly or indirectly to one another. Such joining may be stationary in nature or movable in nature. Such joining may be achieved with the two members and any additional intermediate members being integrally formed as a single unitary body with one another or with the two members or the two members and any additional member being attached to one another. Such joining may be permanent in nature or alternatively may be removable or releasable in nature. As used herein, “rigidly coupled” refers to two components being coupled in a manner such that the components move together in a fixed positional relationship when acted upon by a force.

While the current application recites particular combinations of features in the claims appended hereto, various embodiments of the invention relate to any combination of any of the features described herein whether or not such combination is currently claimed, and any such combination of features may be claimed in this or future applications. Any of the features, elements, or components of any of the exemplary embodiments discussed above may be used alone or in combination with any of the features, elements, or components of any of the other embodiments discussed above.

In various exemplary embodiments, the relative dimensions, including angles, lengths, and radii, as shown in the Figures are to scale. Actual measurements of the Figures will disclose relative dimensions, angles, and proportions of the various exemplary embodiments. Various exemplary embodiments extend to various ranges around the absolute and relative dimensions, angles and proportions that may be determined from the Figures. Various exemplary embodiments include any combination of one or more relative dimensions or angles that may be determined from the Figures. Further, actual dimensions not expressly set out in this description can be determined by using the ratios of dimensions measured in the Figures in combination with the express dimensions set out in this description.

What is claimed is:

1. A banded earmuff, comprising:
a headband comprising a first end, a second end opposite the first end, and a central portion extending between the first end and the second end, the central portion comprising:
a first outer arched segment extending from the first end to the second end;
a second outer arched segment opposite the first outer arched segment and extending from the first end to the second end, wherein the second outer arched segment is spaced apart from the first outer arched segment; and
at least one opening defined along a length of the headband between the first end, the second end, the first outer arched segment, and the second outer arched segment;
a first ear cup coupled to the first end; and
a second ear cup coupled to the second end; and
wherein the headband provides a compressive force to the first ear cup and the second ear cup to bias the first ear cup and the second ear cup towards each other.

2. The banded earmuff of claim 1, wherein the headband is formed from a single, continuous, contiguous piece of material.

3. The banded earmuff of claim 1, wherein the central portion further comprises a first base segment at the first end extending between the first outer arched segment and the second outer arched segment, and a second base segment at the second end extending between the first outer arched segment and the second outer arched segment.

4. The banded earmuff of claim 3, wherein the at least one opening extends uninterrupted from the first base segment to the second base segment.

5. The banded earmuff of claim 1, wherein the headband further comprises a support extending between the first outer arched segment and the second outer arched segment, and wherein the support is positioned such that it overlaps at least a portion of the at least one opening.

6. The banded earmuff of claim 5, wherein a first wire segment is coupled to a first side of the support and a second wire segment is coupled to a second side of the support opposite the first side.

7. The banded earmuff of claim 6, wherein the first wire segment is retained within the first outer arched segment, and wherein the second wire segment is retained within the second outer arched segment.

8. The banded earmuff of claim 1, wherein the compressive force provided by the headband is at least 9.81N.

9. The banded earmuff of claim 1, wherein the first ear cup comprises an outer shell and a pad, and wherein the pad is formed from acoustic foam.

10. A hearing protector, comprising:
a headband comprising:
a first end;
a second end opposite the first end;
a first outer arched segment extending along a length of the headband from the first end to the second end;
a second outer arched segment spaced apart from the first outer arched segment and extending along the length of the headband from the first end to the second end; and
an opening defined between the first end, the second end, the first outer arched segment, and the second outer arched segment;
a first earmuff coupled to the first end of the headband, the first earmuff comprising a first pad; and
a second earmuff coupled to the second end of the headband, the second earmuff comprising a second pad; and
wherein the headband is configured to bias the first earmuff and the second earmuff towards the head of a user when the hearing protector is worn.

11. The hearing protector of claim 10, wherein the first pad and the second pad are formed from acoustic foam.

12. The hearing protector of claim 10, wherein the opening extends uninterrupted along the length of the headband between the first end and the second end.

13. The hearing protector of claim 10, wherein the headband is formed from a single, continuous, contiguous piece of material.

14. The hearing protector of claim 10, wherein the opening is a first opening, wherein the headband further comprises a central band coupled to the first outer arched segment and the second outer arched segment, wherein the central band comprises a second opening.

15. The hearing protector of claim 14, wherein the second opening overlaps with the first opening such that the second opening is positioned between the first end, the second end, the first outer arched segment, and the second outer arched segment.

16. A headband for protective hearing equipment, the headband comprising:
a first end;
a second end opposite the first end;
a first outer arched segment extending from the first end to the second end;
a second outer arched segment opposite the first outer arched segment and extending from the first end to the second end;
a first base segment at the first end extending between the first outer arched segment and the second outer arched segment; and
a second base segment at the second end extending between the first outer arched segment and the second outer arched segment; and
an opening defined between the first base segment, the second base segment, the first outer arched segment, and the second outer arched segment, the opening comprising a first end terminating at the first base segment and a second end opposite the first end and terminating at the second base segment, wherein at least a portion of the opening is uninterrupted between the first end of the opening and the second end of the opening.

17. The headband of claim 16, wherein the headband is formed from a single, continuous, contiguous piece of metal material.

18. The headband of claim 16, further comprising a central band extending between the first outer arched segment and the second outer arched segment, and wherein the central band is positioned such that it overlaps at least a first portion of the first end of the opening and a second portion of the second end of the opening.

19. The headband of claim 18, further comprising a first wire segment coupled to a first side of the central band and a second wire segment coupled to a second side of the central band, wherein the first wire segment is retained within the first outer arched segment, and wherein the second wire segment is retained within the second outer arched segment.

20. The headband of claim 16, wherein a cross-section of the headband defined along a plane extending perpendicular to a width of the headband has a sectional moment of inertia of at least 0.63 mm⁴ and at most 0.7 mm⁴.
